(19) United States
(12) Patent Application Publication
(10) Pub. No.: US 2014/0114086 A1
(43) Pub. Date: Apr. 24, 2014

(54) Clinprost, Isocarbacyclin And Analogs Thereof And Methods Of Making The Same

(71) Applicant: The University Of North Carolina At Greensboro, Greensboro, NC (US)

(72) Inventor: Mitchell Croatt, Greensboro, NC (US)

(21) Appl. No.: 14/058,520

(22) Filed: Oct. 21, 2013

Related U.S. Application Data
(60) Provisional application No. 61/716,869, filed on Oct. 22, 2012.

(51) Int. Cl. C07C 67/30; C07C 69/608

(52) U.S. Cl. CPC C07C 67/30; C07C 69/608
USPC 560/119

(57) ABSTRACT

In one aspect, methods of synthesizing clinprost, isocarbacyclin and analogs thereof are described herein which, in some embodiments, permit an abbreviated synthetic pathway in comparison to one or more prior synthetic methods. By providing a compact synthetic scheme, methods described herein can reduce cost, waste and time of clinprost and isocarbacyclin synthesis while facilitating the development and investigation of analogs of these compounds.

### CROSS REFERENCE TO RELATED APPLICATION

This application claims priority pursuant to 35 U.S.C. §119(e) to U.S. Provisional Patent Application Ser. No. 61/716,869, filed on Oct. 22, 2012, which is hereby incorporated by reference in its entirety.

### FIELD

The present invention relates to clinprost and isocarbacyclin analogs and, in particular, to methods of synthesizing clinprost, isocarbacyclin and analogs thereof.

### BACKGROUND

By demonstrating platelet aggregation inhibiting properties in conjunction with the ability to serve as a potent endogenous vasodilator, prostacyclin has received significant pharmaceutical attention and investigation. Prostacyclin, however, has inherent instability stemming from the presence of vinyl ether and carboxylic acid moieties. In view of this instability, analogs of prostacyclin have been developed into pharmaceutical compositions for indications related to vasodilation, including the treatment of hypertension. For example, carbacyclin and related structures of iloprost, beraprost and treprostinil have been developed for such indications. Further, clinprost, the methyl ester of isocarbacyclin, has also been synthesized.

The synthesis of isocarbacylcin and clinprost, however, is difficult, often requiring greater than 20 steps from commercially available reagents. Such synthetic difficulty has limited the facile development and investigation of analogs of isocarbacyclin and clinprost for various indications.

### SUMMARY

In one aspect, methods of synthesizing clinprost, isocarbacyclin and analogs thereof are described herein which, in some embodiments, permit an abbreviated synthetic pathway in comparison to one or more prior synthetic methods. By providing a compact synthetic scheme, methods described herein can reduce cost and time of clinprost and isocarbacyclin synthesis while facilitating the development and investigation of analogs of these compounds.

A method described herein of synthesizing clinprost, isocarbacyclin or an analog thereof, in some embodiments, comprises providing an ester of formula (1):

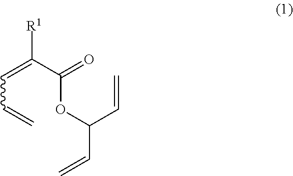

and performing a decarboxylation with concomitant allylic transposition to provide a reaction product mixture comprising a tetraene of formula (2):

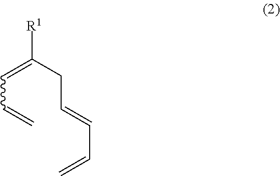

wherein R¹ is selected from the group consisting of —C(O)OR³, -alkyl-C(O)OR³ and -alkyl-OR³, wherein R³ is hydrogen or alkyl. Cyclocarbonylation is performed on the tetraene of formula (2) followed by reduction of the resulting ketone to provide a compound of formula (3):

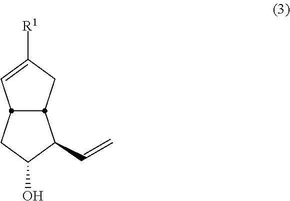

The compound of formula (3) undergoes cross-metathesis with an alkene to provide a compound of formula (4):

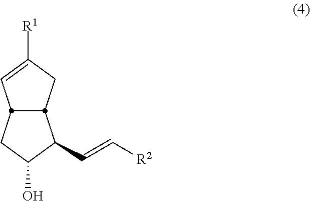

wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.

In another aspect, a method described herein of synthesizing clinprost or isocarbacyclin analogs comprises providing an ester of formula (6):

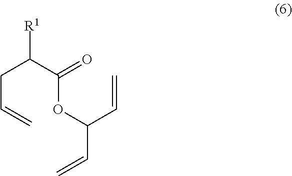

and performing a decarboxylation with concomitant allylic transposition to provide a reaction product mixture comprising a tetraene of formula (7):

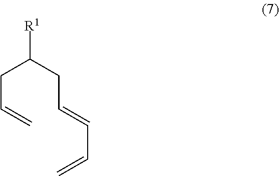

wherein R¹ is selected from the group consisting of —C(O)OR³, -alkyl-C(O)OR³ and -alkyl-OR³, wherein R³ is hydrogen or alkyl. Cyclocarbonylation is performed on the tetraene of formula (7) followed by reduction of the resulting ketone to provide a compound of formula (8):

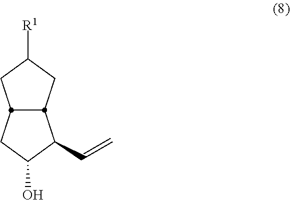

The compound of formula (8) undergoes cross-metathesis with an alkene to provide a compound of formula (9):

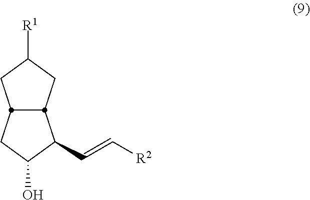

wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.

In another aspect, analogs of clinprost or isocarbacyclin are provided herein which, in some cases, can be used in pharmaceutical compositions for indications related to vasodilation, including the treatment of hypertension. In some embodiments such an analog is of formula (4):

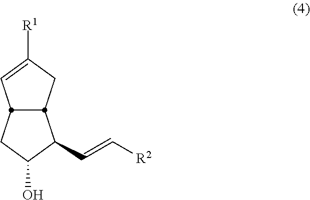

wherein R¹ is selected from the group consisting of —C(O)OR³, -alkyl-C(O)OR³ and -alkyl-OR³, wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃. In one embodiment, for example, R¹ is -alkyl-C(O)OR³ and R² is alkyl. Further, in some such cases, R³ is hydrogen.

In other embodiments, an analog of clinprost or isocarbacyclin described herein is of formula (9):

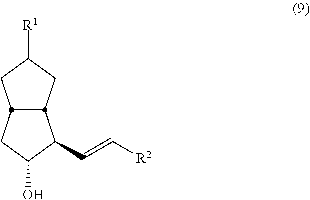

wherein R¹ is selected from the group consisting of —C(O)OR³, -alkyl-C(O)OR³ and -alkyl-OR³, wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃. In one embodiment, for example, R¹ is -alkyl-C(O)OR³ and R² is alkyl.

These and other embodiments are described in greater detail in the detailed description which follows.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### DEFINITIONS

In the structural formulas provided herein and throughout the present specification, the following terms have the indicated meaning:

The term “optionally substituted” means that the group in question is either unsubstituted or substituted with one or more of the substituents specified. When the groups in question are substituted with more than one substituent, the substituent may be the same or different.

The term “alkyl” as used herein, alone or in combination, refers to a straight or branched chain saturated monovalent hydrocarbon radical. In some embodiments, for example, alkyl groups include, but are not limited to, methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-pentyl, n-hexyl, n-heptyl, n-octyl, 1,2-dimethylpropyl, 2,2-dimethylpropyl, 1,2,2-trimethylpropyl and the like.

The term “alkenyl” as used herein, alone or in combination, refers to a straight or branched chain monovalent hydrocarbon radical containing at least one carbon-carbon double bond. In some embodiments, for example, alkenyl groups include, but are not limited to, allyl, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “cycloalkyl” as used herein, alone or in combination, refers to a non-aromatic monovalent hydrocarbon radical ring having from three to twelve carbon atoms, and optionally with one or more degrees of unsaturation. For example, in some embodiments, cycloalkyl groups include, but are not limited to, cyclopropyl, cyclobutyl, cyclopentyl, cyclohexyl, cyclohexenyl, cycloheptyl, cycloheptenyl, cyclooctyl and the like.

The term “heterocyclic” or the term “heterocyclyl” as used herein, alone or in combination, refers to a three to twelve membered ring having atoms of at least two different elements. For example, in some embodiments, a heterocyclic group comprises a hydrocarbon ring containing one or more heteroatomic substitutions selected from the group consisting of N, O and S. A heterocyclic ring may be optionally fused to one or more of another heterocyclic ring(s), cycloalkyl ring(s) and/or aryl groups.

The term “aryl” as used herein refers to a carbocyclic aromatic ring radical or to an aromatic ring system radical. Aryl is also intended to include the partially hydrogenated derivatives of the carbocyclic systems.

The term “heteroaryl” as used herein, alone or in combination, refers to an aromatic ring radical with, for instance, 5 to 7 member atoms or to an aromatic ring system radical with, for instance, from 7 to 18 member atoms containing one or more heteroatoms selected from the group consisting of N, O and S.

The term “alkoxy” as used herein, alone or in combination, refers to the monovalent radical RO—, where R is alkyl or alkenyl defined above. For example, alkoxy groups include, but are not limited to, methoxy, ethoxy, n-propoxy, butoxy, sec-butoxy, tert-butoxy, pentoxy, isopentoxy, hexoxy and the like.

In one aspect, methods of synthesizing clinprost, isocarbacyclin and analogs thereof are described herein which, in some embodiments, permit an abbreviated synthetic pathway in comparison to one or more prior synthetic methods. By providing a compact synthetic scheme, methods described herein can reduce cost and time of clinprost and isocarbacyclin synthesis while facilitating the development and investigation of analogs of these compounds.

### I. Methods of Synthesizing Clinprost and Analogs Thereof

A method described herein of synthesizing isocarbacyclin, clinprost or an analog thereof, in some embodiments, comprises providing an ester of formula (1):

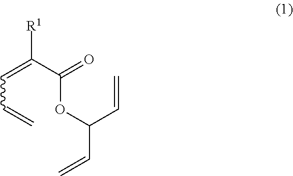

and performing a decarboxylation with concomitant allylic transposition to provide a reaction product mixture comprising a tetraene of formula (2):

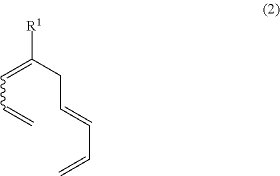

wherein R¹ is selected from the group consisting of -alkyl-C(O)OR³ and -alkyl-OR³, wherein R³ is hydrogen or alkyl. Cyclocarbonylation is performed on the tetraene of formula (2) followed by reduction of the resulting ketone in situ to provide a compound of formula (3):

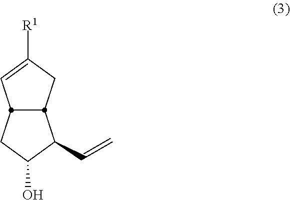

The compound of formula (3) undergoes cross-metathesis with an alkene to provide a compound of formula (4):

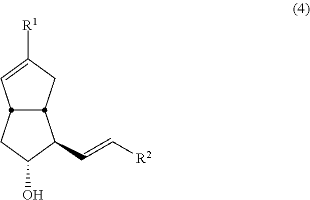

wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.

Turning now to specific steps, synthetic methods described herein, in some embodiments, comprise providing an ester of formula (1):

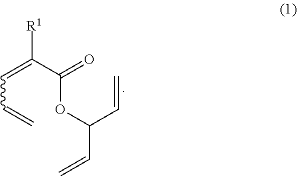

An ester of formula (1), in some embodiments, is provided by esterification of a carboxylic acid with an alcohol of formula (5) in conjunction with deprotonation and aldol reactions.

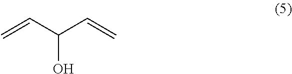

Carboxylic acids suitable for esterification with the alcohol of formula (5) can include monocarboxylic acids and dicarboxylic acids. For example, in one embodiment, a carboxylic acid for esterification with the alcohol of formula 5 is pimelic acid. Additionally, deprotonation and aldol reactions can be administered with lithium bis(trimethylsilyl)amide (LiHMDS) and acrolein respectively. Further, depending on the conditions for mesylation and elimination, the E/Z of ratio the ester of formula (1) can be modified. As both isomers can lead to a compound of formula (4), there is no requirement for separation of diastereomers during stage(s) in the synthetic pathway. In some embodiments, for example, an ester of formula (1) is provided according to Scheme 1.

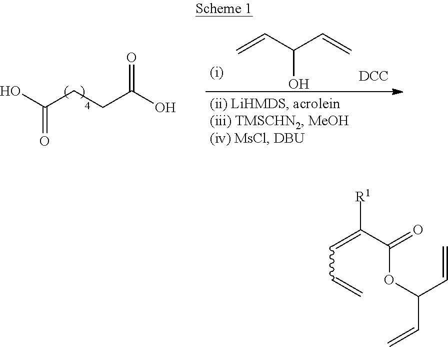

As described herein, the ester of formula (1) undergoes intramolecular decarboxylative allylation to provide a reaction product mixture comprising the tetraene of formula (2):

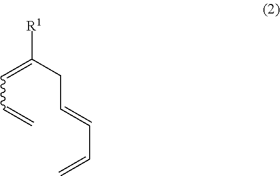

The decarboxylative allylation can be administered with suitable transition metal catalyst, such as palladium catalyst. In some embodiments, for example, the intramolecular decarboxylative allylation proceeds according to Scheme 2.

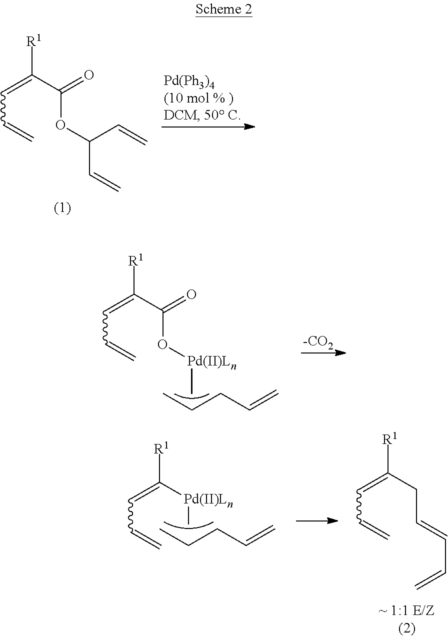

Although decarboxylative allylations are known for allylic esters where the alpha position of the ester is stabilized, typically with an electron withdrawing group, decarboxylations of either the present bis-allylic ester or where a non-stabilized Pd—C bond is formed are unprecedented.

The tetraene of formula (2) undergoes a cyclocarbonylation reaction in the presence of a transition metal catalyst followed by reduction of the resulting ketone to provide the compound of formula (3) herein. In some embodiments, the reduction of the ketone is accomplished in situ. Both isomers (E/Z) of the tetraene (2) are reactive for the cyclocarbonylation reaction, with the cis-isomer demonstrating significantly higher reactivity. However, the trans-isomer can be resubjected to a more reactive transition metal catalyst to provide the compound of formula (3). In some embodiments, for example, the cyclocarbonylation reaction and subsequent ketone reduction proceed according to Scheme 3. The compound of formula (3) can be purified from other reaction products by chromatographic techniques, such as silica gel chromatography.

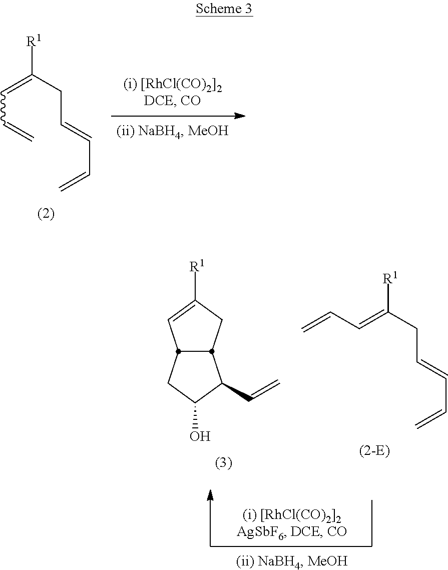

The compound of formula (3) is operable to undergo cross-metathesis with a variety of alkenes for the production of a compound of formula (4).

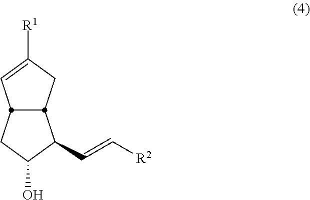

Silyl ether can be used to protect hydroxyl functionalities on the alkene during the cross-metathesis. In some embodiments, for example, tert-butyldimethyl(non-1-en-3-yloxy)silane [TBS] is used as a protecting group. Protected hydroxyl functionalities can be regenerated by treatment with tetrabutylamoniun fluoride (TBAF).

In some embodiments, for example, cross-metathesis proceeds according to Scheme 4.

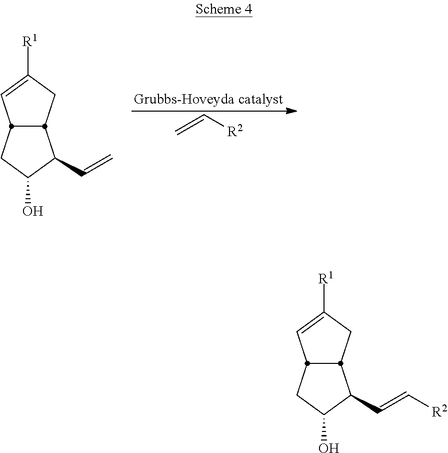

As described herein, R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃. In some embodiments, for example, an alkene for cross-metathesis with the compound of formula (3) in the production of a compound of formula (4) is selected from any of the following alkenes:

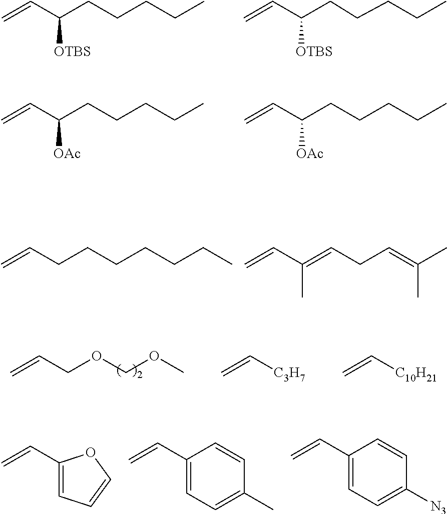

Further, methods described herein, in some embodiments, can be employed to provide clinprost, isocarbacyclin or compound of formula (4) described in Section II herein.

In another aspect, a method described herein of synthesizing clinprost or isocarbacyclin analogs comprises providing an ester of formula (6):

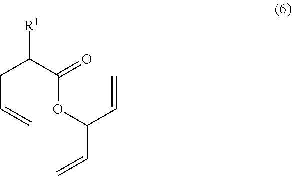

and performing a decarboxylation with concomitant allylic transposition to provide a reaction product mixture comprising a tetraene of formula (7):

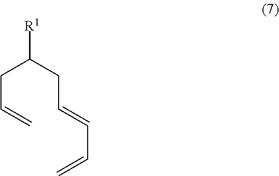

wherein R¹ is selected from the group consisting of —C(O)OR³, -alkyl-C(O)OR³ and -alkyl-OR³, wherein R³ is hydrogen or alkyl. Cyclocarbonylation is performed on the tetraene of formula (7) followed by reduction of the resulting ketone to provide a compound of formula (8):

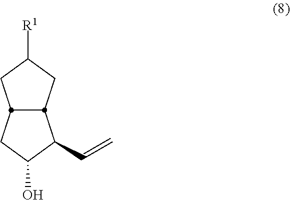

The compound of formula (8) undergoes cross-metathesis with an alkene to provide a compound of formula (9):

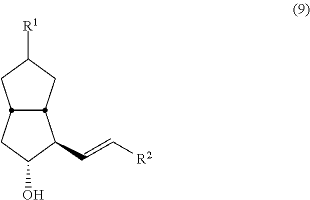

wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.

Turning now to specific steps, synthetic methods described herein, in some embodiments, comprise providing an ester of formula (6):

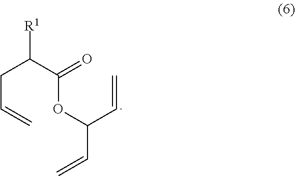

An ester of formula (6), in some embodiments, is provided by esterification of a carboxylic acid with an alcohol of formula (5) in conjunction with deprotonation and aldol reactions, in a manner similar to that described hereinabove regarding the ester of formula (1).

As described herein, the ester of formula (6) undergoes decarboxylation with concomitant allylic transposition to provide a reaction product mixture comprising the tetraene of formula (7):

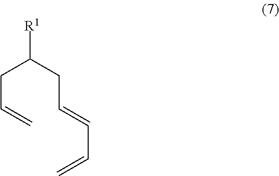

The decarboxylative allylation, in some embodiments, can be administered with suitable transition metal catalyst, such as palladium catalyst.

The tetraene of formula (7) undergoes a cyclocarbonylation reaction followed by reduction of the resulting ketone to provide the compound of formula (8) herein. In some embodiments, the cyclocarbonylation is carried out in the presence of a transition metal catalyst, such as a rhodium catalyst. The reduction of the ketone, in some embodiments, is accomplished in situ. The compound of formula (8) can be purified from other reaction products by chromatographic techniques, such as silica gel chromatography.

The compound of formula (8) is operable to undergo cross-metathesis with a variety of alkenes for the production of a compound of formula (9).

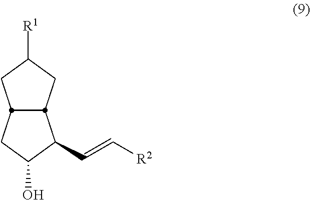

The alkenes can include any of the alkenes described hereinabove regarding the production of a compound of formula (4). In addition, as provided hereinabove, silyl ether can be used to protect hydroxyl functionalities on the alkene during the cross-metathesis. In some embodiments, for example, tert-butyldimethyl(non-1-en-3-yloxy)silane [TBS] is used as a protecting group. Protected hydroxyl functionalities can be regenerated by treatment with tetrabutylamoniun fluoride (TBAF).

In some embodiments, cross-metathesis proceeds according to a catalytic reaction. Any metathesis catalyst not inconsistent with the objectives of the present invention may be used. Non-limiting examples of metathesis catalysts include ruthenium, molybdenum, and tungsten catalysts.

Further, methods described herein, in some embodiments, can be employed to provide clinprost or isocarbacyclin analogs of formula (9) described in Section II herein.

### II. Analogs of Clinprost and Isocarbacyclin

In another aspect, analogs of clinprost and/or isocarbacyclin are provided herein.

In some embodiments an analog is of formula (4):

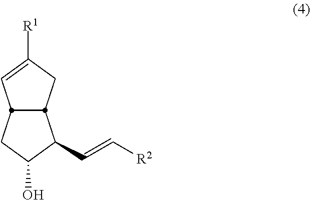

wherein R¹ is selected from the group consisting of —C(O)OR³, -alkyl-C(O)OR³ and -alkyl-OR³, wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃. In one embodiment, for example, R¹ is -alkyl-C(O)OR³ and R² is alkyl.

In some embodiments, an analog of clinprost or isocarbacyclin of formula (4) is selected from any of the following compounds:

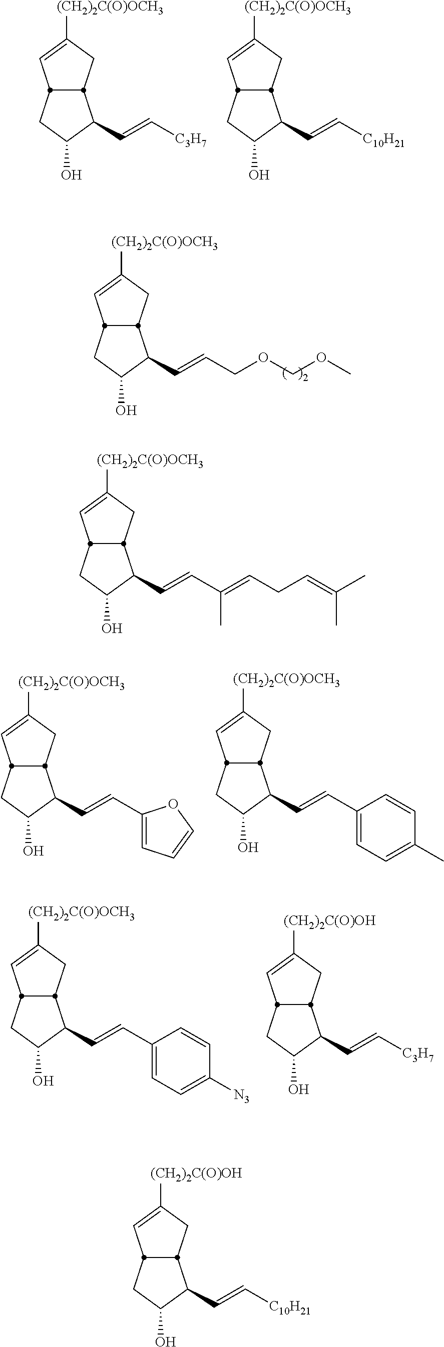

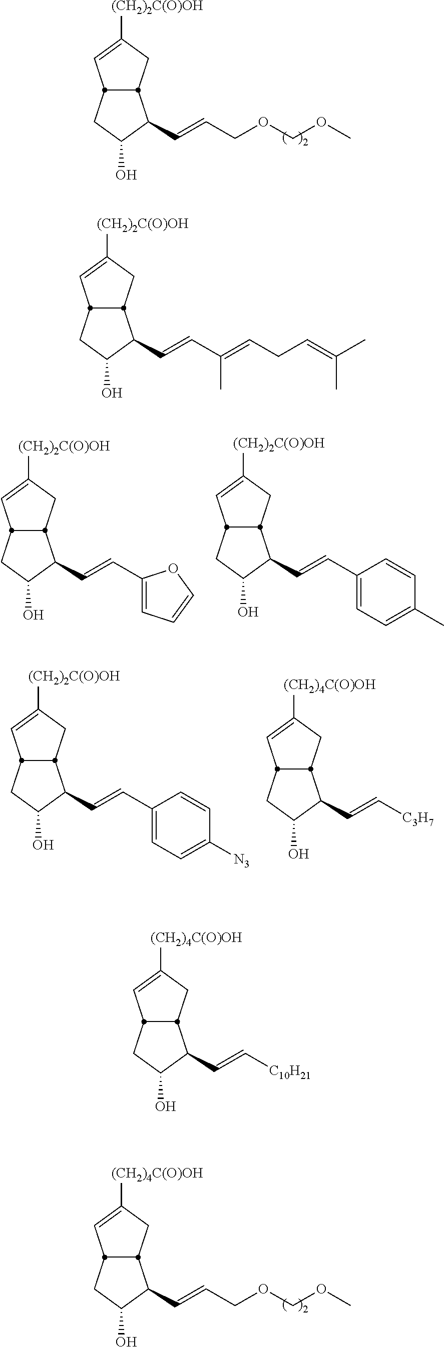

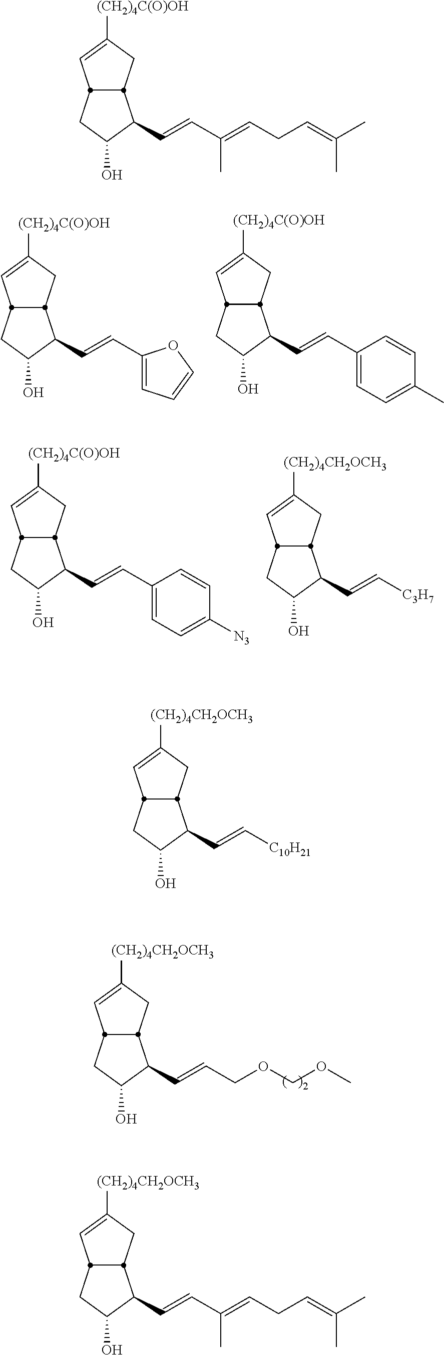

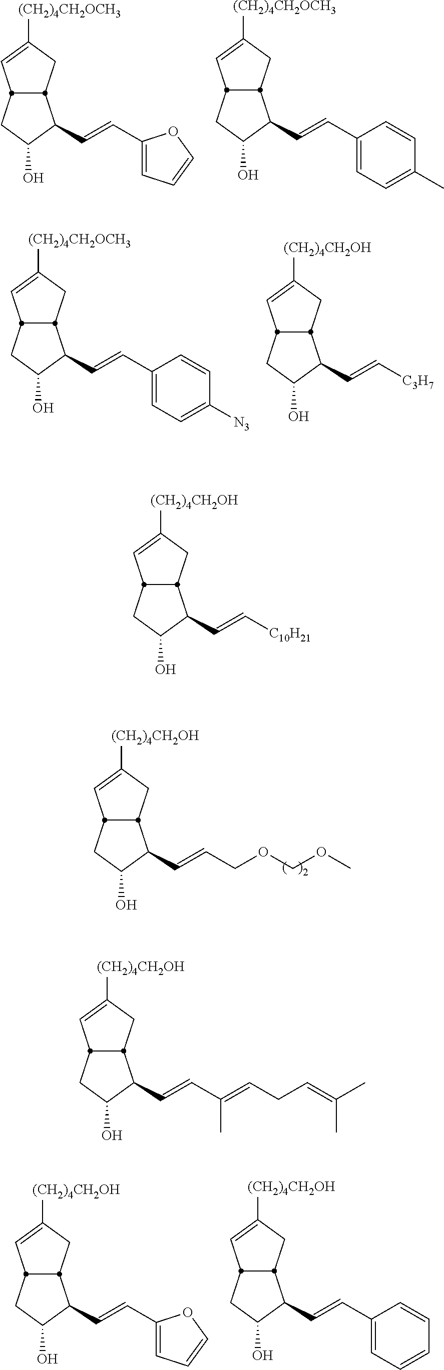

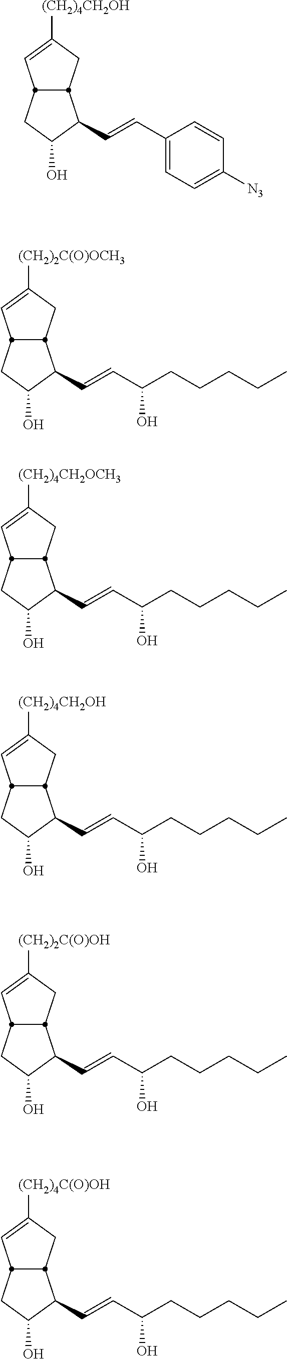

In another aspect, an analog of clinprost or isocarbacyclin described herein is of formula (9):

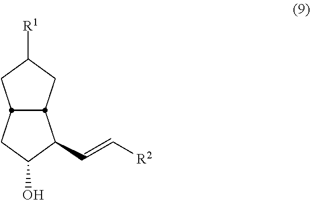

wherein R¹ is selected from the group consisting of —C(O)OR³, -alkyl-C(O)OR³ and -alkyl-OR³, wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃. In one embodiment, for example, R¹ is -alkyl-C(O)OR³ and R² is alkyl. Further, in some such cases, R³ is hydrogen.

In some embodiments, an analog of clinprost or isocarbacyclin of formula (9) is selected from any of the following compounds:

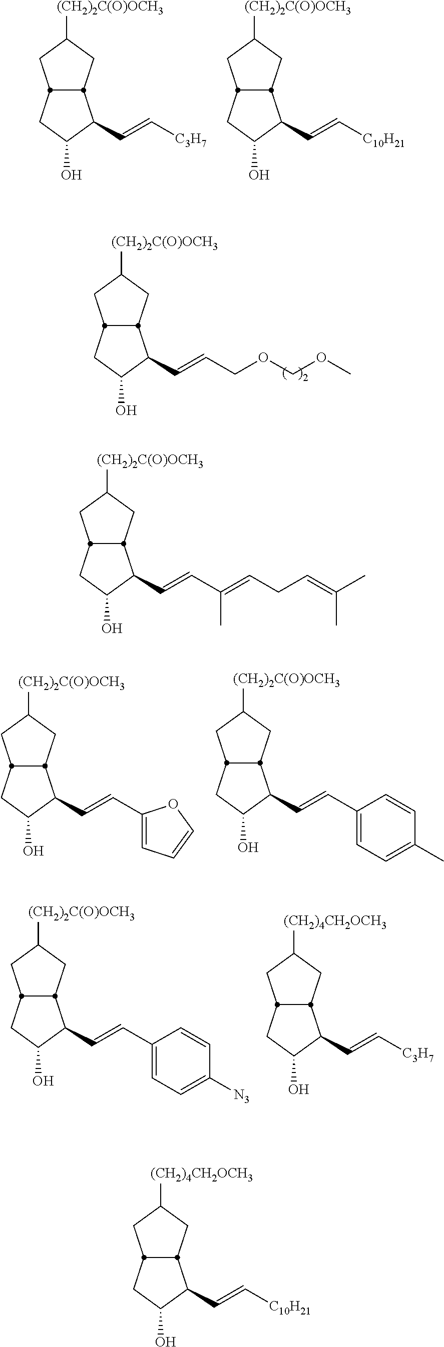

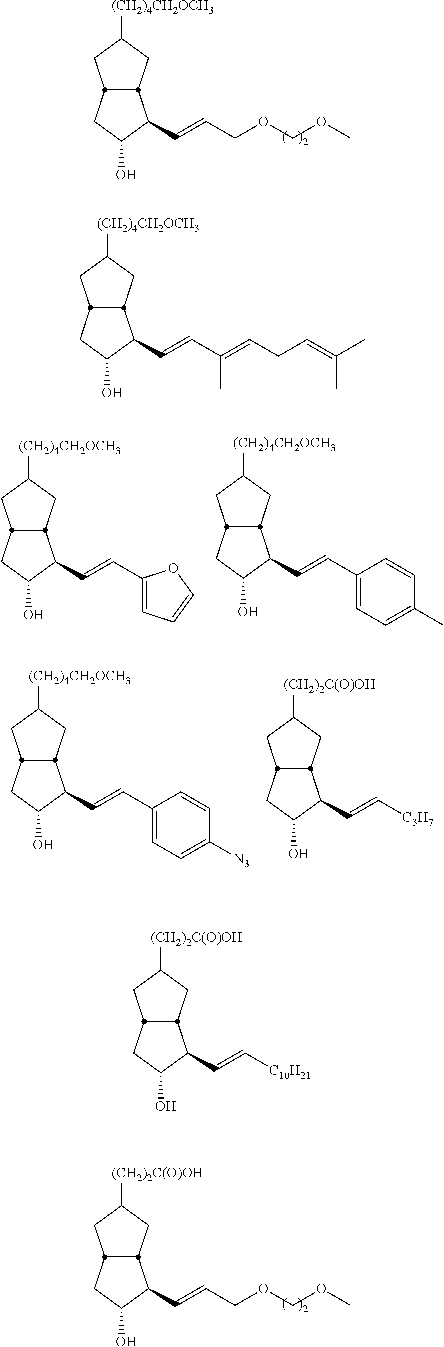

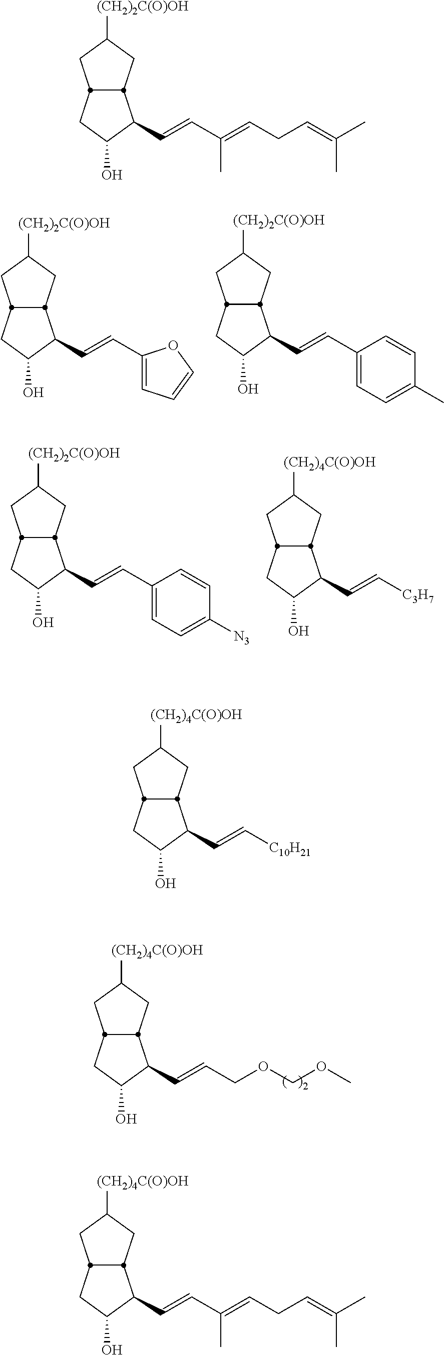

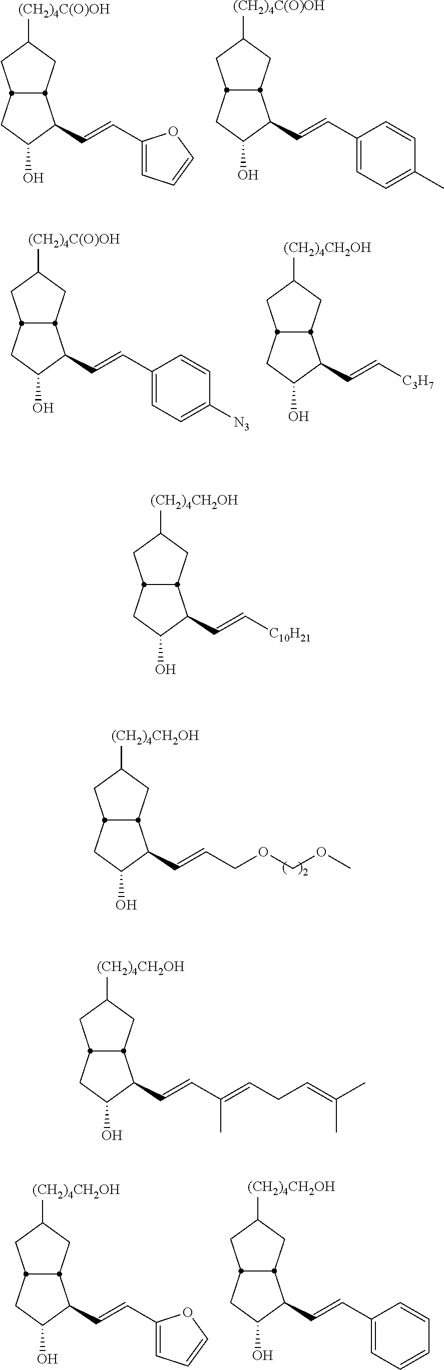

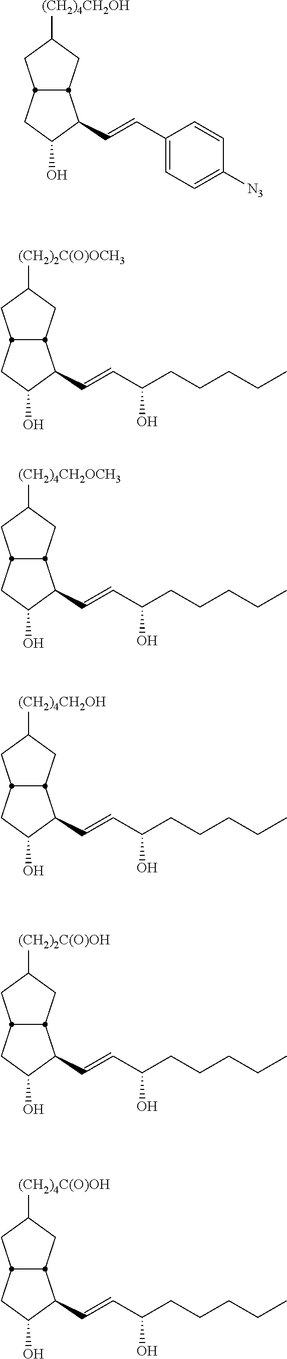

These and other embodiments are further illustrated in the following non-limiting examples.

### General Information for Examples

All anhydrous reactions were performed with dry solvents in oven dried glassware under a nitrogen atmosphere. Unless otherwise noted, all solvents and reagents were obtained from commercial sources and used without further purification.

Chromatographic purification was performed using silica gel (60 Å, 32-63 μm). NMR spectra were recorded using a Bruker AVANCE DRX 300 spectrometer (300 MHz for ¹H), JEOL ECA spectrometer (500 MHz for ¹H, 125 MHz for ¹³C) and an Agilent 400-MR spectrometer equipped with a 1H/19F/X 5 mm PFG Broadband probe (400 MHz for ¹H and 100 MHz for ¹³C). Coupling constants, J, are reported in hertz (Hz) and multiplicities are listed as singlet (s), doublet (d), triplet (t), doublet of doublets (dd), triplet of triplets (tt), quintet (quint), multiplet (m), etc. IR data was obtained with a Perkin Elmer FTIR spectrometer with ATR sampling accessory with frequencies reported in cm⁻¹. High Resolution Mass Spectra were acquired on a ThermoFisher Scientific LTQ Orbitrap XL MS system.

It is to be understood that the following examples illustrate the synthesis of only some selected chemical species and chemical products described herein. For instance, the following examples illustrate the synthesis of some selected species having particular R¹ and R² groups described herein. However, as understood by one of ordinary skill in the art, other chemical species and chemical products having different R¹ and/or R² groups described herein can be made in a similar manner by using analogous reagents in place of one or more reagents described in one or more of Examples 1-14. In some embodiments, for example, it is possible to replace a dicarboxylic acid or alkene described in Example 1 or Example 7, respectively, with a different dicarboxylic acid or alkene to provide a product described herein having R¹ and R² groups that differ from those described in the specific examples below.

Moreover, in some cases, it is also possible to make other chemical species and chemical products described herein by carrying out an additional step that may not be described in Examples 1-14, such as an additional hydrolysis step. For instance, in some of the following examples, R¹ is —(CH₂)₄C(O)OMe. However, as understood by one of ordinary skill in the art, a final product or intermediate species in which R¹ is —(CH₂)₄C(O)OH can be provided, if desired, by hydrolyzing the ester of —(CH₂)₄C(O)OMe to form the carboxylic acid analogue.

### Example 1

### Esterification of Dicarboxylic Acid

A solution of DCC (2.45 g, 11.9 mmol) in THF (15 mL) was added to a solution of pimelic acid (9.54 g, 59.4 mmol), 1,4-pentadien-3-ol (1.16 mL, 11.9 mmol), and DMAP (145 mg, 1.19 mmol) in THF (90 mL) slowly via an additional funnel over 3 hours. After 2 days, the reaction was filtered through celite and washed with THF. Silica gel was added to the concentrated mixture and then the solvent was removed after which the dry powder was added to a silica gel column. The product was purified (7:3, hexanes, EtOAc) to yield 7-oxo-7-(penta-1,4-dien-3-yloxy)heptanoic acid (2.1 g, 78%).

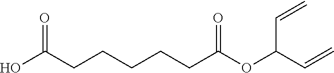 [SMILES: C=CC(C=C)OC(=O)CCCCCC(=O)O]

¹H NMR (500 MHz, CDCl₃) δ=11.34-11.00 (br s, 1H), 5.88-5.79 (m, 2H), 5.74-5.69 (m, 1H), 5.34-5.21 (m, 4H), 2.36 (dt, J=1.7, 7.4 Hz, 4H), 1.67 (qd, J=7.4, 14.6 Hz, 4H), 1.44-1.35 (m, 2H) ppm.

¹³C NMR (125 MHz, CDCl₃) δ=179.5, 172.4, 135.1 (2C), 117.4 (2C), 74.9, 34.2, 33.7, 28.4, 24.5, 24.3 ppm.

HRMS (ESI) C₁₂H₁₈O₄ [M+Na]⁺, calculated: 249.1103, found: 249.1098.

IR (neat) 2935, 2866, 1732, 1705, 1641, 1170, 925 cm⁻¹.

### Example 2

### Deprotonation and Aldol Reaction

To a solution of LiHMDS (13.2 mL, 13.2 mmol) in THF (12 mL) at −78° C. 7-oxo-7-(penta-1,4-dien-3-yloxy)heptanoic acid of Example 1 (1.0 g, 4.4 mmol) in THF (6 mL) was added slowly. After stirring for 15 minutes, the reaction was transferred via cannula quickly to a mixture of acrolein (2.9 mL, 44 mmol) in THF (22 mL) cooled to −78° C. Additional THF (4 mL) was transferred as a wash. During the transfer, the reaction turned from clear to blue to green. After 4 minutes of stirring, the reaction was quenched by addition of saturated aqueous NH₄Cl (10 mL) and allowed to warm to room temperature. After 2 extractions with EtOAc, a 1M HCl solution was added to the aqueous layer until pH˜3. This addition was followed by 2 more extractions with EtOAc. The combined organic layers were dried using Na₂SO₄ and filtered if there were solids. They were then concentrated and purified via silica gel chromatography (65:35, hexanes, EtOAc) which yielded 7-hydroxy-6-((penta-1,4-dien-3-yloxy)carbonyl)non-8-enoic acid (0.662 g, 53%) as a yellow oil. Two isomers, diastereomers A and B, are produced.

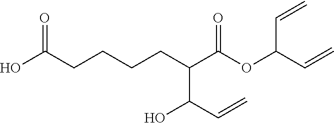 [SMILES: C=CC(C=C)OC(=O)C(CCCCC(=O)O)C(O)C=C]

### Diastereomer A:

¹H NMR (500 MHz, CDCl₃) δ=5.89-5.80 (m, 3H), 5.78-5.72 (m, 1H), 5.36-5.18 (m, 6H), 4.24 (t, J=6.3 Hz, 1H), 2.59-2.51 (m, 1H), 2.35 (t, J=7.4 Hz, 2H), 1.79-1.58 (m, 4H), 1.47-1.30 (m, 2H) ppm.

¹³C NMR (125 MHz, CDCl₃) δ=179.4, 173.8, 138.2, 134.7 (2C), 118.0, 117.8, 116.8, 75.4, 73.6, 50.9, 33.7, 28.8, 26.5, 24.4 ppm.

HRMS (ESI) C₁₅H₂₂O₅ [M+Na]⁺, calculated: 305.1365, found: 305.1356.

IR (neat) 3500, 2927, 1707, 1643, 926 cm⁻¹.

### Diastereomer B:

¹H NMR (500 MHz, CDCl₃) δ=5.89-5.80 (m, 3H), 5.78-5.72 (m, 1H), 5.36-5.18 (m, 6H), 4.35 (t, J=5.7 Hz, 1H), 2.59-2.51 (m, 1H), 2.35 (t, J=7.4 Hz, 2H), 1.79-1.58 (m, 4H), 1.47-1.30 (m, 2H) ppm.

¹³C NMR (125 MHz, CDCl₃) δ=179.4, 173.4, 137.3, 134.7 (2C), 118.03, 117.96, 116.7, 75.5, 73.1, 50.9, 33.7, 26.9, 26.8, 24.4 ppm.

HRMS (ESI) C₁₅H₂₂O₅ [M+Na]⁺, calculated: 305.1365, found: 305.1356.

IR (neat) 3500, 2927, 1707, 1643, 926 cm⁻¹.

### Example 3

### Diester Synthesis

A solution of 7-hydroxy-6-((penta-1,4-dien-3-yloxy)carbonyl)non-8-enoic acid (705 mg, 2.5 mmol) in MeOH (25 mL) was cooled to 0° C. and then trimethylsilyldiazomethane (6.88 mL, 13.75 mmol) was added slowly. After 10 minutes of stirring, N₂ was bubbled through the reaction for 20 minutes and then the reaction was concentrated before purification via silica gel chromatography (8:2, hexanes, EtOAc). Purification yielded 7-methyl 1-penta-1,4-dien-3-yl 2-(1-hydroxyallyl)heptanedioate (688 mg, 93%) as a colorless oil. Two isomers are produced, diastereomer A and B. Diastereomeric ratio is 1:1 with diastereomer A eluting early.

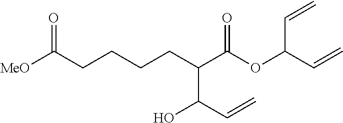 [SMILES: C=CC(C=C)OC(=O)C(CCCCC(=O)OC)C(O)C=C]

### Diastereomer A:

¹H NMR (500 MHz, CDCl₃) δ=5.89-5.79 (m, 3H), 5.36-5.26 (m, 3H), 5.26-5.21 (m, 2H), 5.21-5.16 (m, 1H), 4.23 (tq, Jₜ=1.2 Hz, J_{q}=6.9 Hz, 1H), 3.66 (s, 3H), 2.57 (d, J=6.9 Hz, 1H, OH), 2.57-2.51 (m, 1H), 2.30 (m, 2H), 1.78-1.58 (m, 4H), 1.44-1.21 (m, 2H) ppm.

¹³C NMR (125 MHz, CDCl₃) δ=174.0, 173.4, 137.4, 134.7 (2C), 118.0, 117.9, 116.7, 75.4, 73.1, 51.5, 50.9, 33.8, 27.0, 26.8, 24.7 ppm.

HRMS (ESI) C₁₆H₂₄O₅ [M+H]⁺, calculated: 297.1697, found: 297.1693.

IR (neat) 3510, 2953, 1731, 1643, 926 cm⁻¹.

### Diastereomer B:

¹H NMR (500 MHz, CDCl₃) δ=5.89-5.79 (m, 3H), 5.78-5.72 (m, 1H), 5.36-5.26 (m, 3H), 5.26-5.21 (m, 2H), 5.21-5.16 (m, 1H), 4.35 (tq, Jₜ=1.2 Hz, J_{q}=5.2 Hz, 1H), 3.66 (s, 3H), 2.57-2.51 (m, 1H), 2.47 (d, J=4.6 Hz, 1H, OH), 2.30 (m, 2H), 1.78-1.58 (m, 4H), 1.44-1.21 (m, 2H) ppm.

¹³C NMR (125 MHz, CDCl₃) δ=173.9, 173.8, 138.3, 134.7 (2C), 118.0, 117.7, 116.7, 75.3, 73.6, 51.5, 50.9, 33.8, 28.9, 26.7, 24.7 ppm.

HRMS (ESI) C₁₆H₂₄O₅ [M+H]⁺, calculated: 297.1697, found: 297.1693.

IR (neat) 3510, 2953, 1731, 1643, 926 cm⁻¹.

### Example 4

### Alcohol Elimination

A solution of 7-methyl 1-penta-1,4-dien-3-yl 2-(1-hydroxyallyl)heptanedioate (494 mg, 1.67 mmol) and triethylamine (0.70 mL 5.01 mL) in CH₂Cl₂ (16.7 mL) was cooled to 0° C. and methane sulfonyl chloride (0.32 mL, 4.18 mmol) was added slowly. After 10 minutes, 1,8-diazobicycloundec-7-ene (1.13 mL, 7.52 mmol) was added and the reaction was allowed to warm to room temperature. After an additional hour, additional 1,8-diazobicycloundec-7-ene (1.13 mL, 7.52 mmol) was added. The reaction was allowed to go for another 18 hours after which the reaction was poured over saturated aqueous NaHCO₃ (6 mL) and the mixture was extracted 3× with CH₂Cl₂. The combined organic layers were dried over Na₂SO₄ and concentrated before being purified using column chromatography (85:15, hexanes:EtOAc) to yield (E/Z)-7-methyl 1-penta-1,4-dien-3-yl 2-allylideneheptanedioate (380 mg, 82%) as a colorless oil.

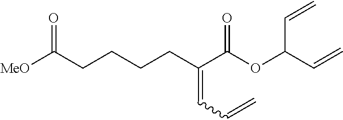 [SMILES: C=CC=C(CCCCC(=O)OC)C(=O)OC(C=C)C=C]

### Diastereomer A:

¹H NMR (500 MHz, CDCl₃) δ=7.22 (d, J=11.7 Hz, 1H), 6.65 (ddd, J=10.0, 11.3, 16.8 Hz, 1H), 5.89 (ddd, J=5.8, 10.3, 16.5 Hz, 2H), 5.79 (tt, J=1.0, 5.8 Hz, 1H), 5.62 (ddd, J=0.7, 1.7, 16.8 Hz, 1H), 5.48 (ddd, J=0.7, 1.7, 10.0 Hz, 1H), 5.33 (dt, J=1.4, 17.2 Hz, 2H), 5.25 (dt, J=1.3, 10 Hz, 2H), 3.66 (s, 3H), 2.45 (t, J=7.6 Hz, 2H), 2.33 (t, J=7.4 Hz, 2H), 1.65 (dd, J=7.6, 15.5 Hz, 2H), 1.52-1.44 (m, 2H) ppm.

¹³C NMR (125 MHz, CDCl₃) δ=174.1, 166.9, 139.2, 135.3 (2C), 132.4, 132.0, 125.1, 117.4 (2C), 75.3, 51.6, 34.0, 29.2, 26.8, 24.9 ppm.

HRMS (ESI) C₁₆H₂₂O₄ [M+H]⁺, calculated: 279.1591, found: 279.1588.

IR (neat) 2950, 2867, 1735, 1706, 1170, 928 cm⁻¹.

### Diastereomer B:

¹H NMR (500 MHz, CDCl₃) δ=7.30-7.22 (m, 1H), 6.39 (d, J=10.9 Hz, 1H), 5.91-5.85 (m, 2H), 5.83-5.80 (m, 1H), 5.41 (dd, J=1.7, 17.2 Hz, 1H), 5.36-5.32 (m, 3H), 5.28-5.24 (m, 2H), 3.67 (s, 3H), 2.37-2.31 (m, 4H), 1.70-1.60 (m, 2H), 1.54-1.44 (m, 2H) ppm.

¹³C NMR (125 MHz, CDCl₃) δ=174.1, 166.9, 139.2, 135.3 (2C), 132.4, 132.0, 125.1, 117.4 (2C), 75.3, 51.6, 34.0, 29.2, 26.8, 24.9 ppm.

HRMS (ESI) C₁₆H₂₂O₄ [M+H]⁺, calculated: 279.1591, found: 279.1588.

IR (neat) 2950, 2867, 1735, 1706, 1170, 928 cm⁻¹.

### Example 5

### Decarboxylation with Allylic Transposition

To a microwave vial, (E/Z)-7-methyl 1-penta-1,4-dien-3-yl 2-allylideneheptanedioate (40 mg, 0.14 mmol) was added in CH₂Cl₂ (2 mL). Tetrakis-(triphenylphosphine) palladium (16.1 mg, 0.014 mmol) was added and the vial was sealed and purged with N₂. The mixture was a dark red/orange color prior to heating. After 24 hours at room temperature, the mixture was a light yellow/orange color, so the reaction was then concentrated and purified via silica gel chromatography (98:2, hexanes:EtOAc) to yield (6E/Z,8E)-methyl 6-allylideneundeca-8,10-dienoate (22.6 mg, 69%) as a yellow oil. Scale-up beyond 40 mg resulted in decreased yields; however, when eight vials were run simultaneously and purified together, the yield remained around 70%.

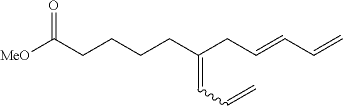 [SMILES: C=CC=C(C/C=C/C=C)CCCCC(=O)OC]

### Diastereomer A:

¹H NMR (500 MHz, CDCl₃) δ=6.74-6.51 (m, 2H), 6.15-6.09 (t, J=10.9 Hz, 1H), 5.88 (d, J=10.9 Hz, 1H), 5.45 (q, J=7.5 Hz, 1H), 5.24 (dd, J=1.4, 16.9 Hz, 1H), 5.18-5.09 (m, 2H), 5.03 (m, 1H), 3.67 (s, 3H), 2.94 (d, J=8.0 Hz, 2H), 2.33 (m, 2H), 2.19 (t, J=1.0 Hz, 2H), 1.68-1.58 (m, 2H), 1.51-1.41 (m, 2H) ppm.

¹³C NMR (125 MHz, CDCl₃) δ=174.1, 141.3, 132.8, 131.8, 130.6, 129.4, 126.7, 117.8, 115.8, 51.5, 35.2, 33.91, 30.5, 27.9, 24.8 ppm.

IR (neat) 3084, 2947, 2862, 1736, 1640, 1592, 1434, 900 cm⁻¹.

### Diastereomer B:

¹H NMR (500 MHz, CDCl₃) δ=6.74-6.51 (m, 2H), 6.09-6.02 (t, J=10.9 Hz, 1H), 5.88 (d, J=10.9 Hz, 1H), 5.35 (q, J=7.5 Hz, 1H), 5.24 (dd, J=1.4, 16.9 Hz, 1H), 5.18-5.09 (m, 2H), 5.03 (m, 1H), 3.67 (s, 3H), 3.06 (d, J=6.9 Hz, 2H), 2.33 (m, 2H), 2.06 (t, J=7.4 Hz, 2H), 1.68-1.58 (m, 2H), 1.51-1.41 (m, 2H) ppm.

¹³C NMR (125 MHz, CDCl₃) δ=174.1, 141.0, 132.7, 131.75, 129.9, 129.7, 126.3, 117.9, 115.9, 51.5, 36.7, 35.2, 33.87, 27.2, 24.6 ppm.

IR (neat) 3084, 2947, 2862, 1736, 1640, 1592, 1434, 900 cm⁻¹.

### Example 6

### Cyclocarbonylation and Reduction

(6E/Z,8E)-Methyl 6-allylideneundeca-8,10-dienoate (40 mg, 0.171 mmol) in dichloroethane (1.7 mL) was added to a dry test tube. [RhCl(CO)₂]₂ (6.6 mg, 0.0171 mmol) was then added before purging thoroughly with CO. A CO filled balloon was used to maintain a constant CO atmosphere and the reaction was heated to 80° C. using an oil bath for 8 hours. The reaction was then cooled to 0° C. and MeOH (2 mL) was added prior to the addition of NaBH₄ (12.9 mg, 0.342 mmol). After 15 minutes, the reaction was poured over water and extracted 2× with EtOAc. The aqueous layer was then acidified (pH=3) by the addition of a 1M HCl solution after which two more extractions were done using EtOAc. The combined organic layers were then washed with brine, extracted 3× with EtOAc, dried using Na₂SO₄ and finally concentrated. Purification via silica gel chromatography (9:1, hexanes, EtOAc) yielded methyl 5-((3aS,5R,6R,6aS)-5-hydroxy-6-vinyl-1,3a,4,5,6,6a-hexahydropentalen-2-yl)pentanoate and methyl 5-((3aR,5S,6S,6aR)-5-hydroxy-6-vinyl-1,3a,4,5,6,6a-hexahydropentalen-2-yl)pentanoate (5.5 mg, 12% or 31% borsm) as a colorless oil.

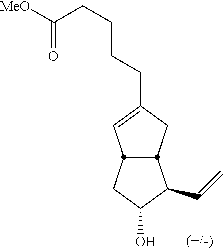 [SMILES: C=C[C@@H]1[C@H]2CC(CCCCC(=O)OC)=C[C@H]2C[C@H]1O]

¹H NMR (500 MHz, CDCl₃) δ=5.73 (ddd, J=8.6, 10.3, 17.2 Hz, 1H), 5.32-5.28 (m, 1H), 5.17-5.08 (m, 2H), 3.84-3.76 (m, 1H), 3.68 (s, 3H), 3.06-2.98 (m, 1H), 2.48-2.41 (m, 1H), 2.39-2.28 (m, 4H), 2.08-2.01 (m, 3H), 1.96 (q, J=9.2 Hz, 1H), 1.71-1.59 (m, 3H), 1.50-1.43 (m, 2H), 1.32 (ddd, J=7.5, 9.7, 16.6 Hz, 1H) ppm.

¹³C NMR (125 MHz, CDCl₃) δ=174.2, 141.4, 139.8, 128.2, 116.6, 59.8, 51.5, 45.7, 44.1, 39.7, 39.6, 33.9, 30.5, 29.7, 27.1, 24.6 ppm.

### Example 7

### (R)-oct-1-en-3-ol and (S)-3-acetoxy-1-octene Synthesis

To a stirred solution of racemic oct-1-en-3-ol (3.1 mL, 20 mmol) in vinyl acetate (33 mL), Novozyme (666 mg) was added. After four hours of stirring, the enzyme was filtered off and washed with diethyl ether. The solution was then concentrated and purified using silica gel chromatography (hexanes:EtOAc) to yield alcohol 12 (1.212 g, 47%) and acetate 13 (1.255 g, 37%) as colorless oils. A 4 hour reaction time was used to achieve 97% ee of the (S)-3-acetoxy-1-octene and an 18 hour reaction time was used to achieve a 99% ee for the (R)-oct-1-en-3-ol . The spectral data matched prior reports: Tetrahedron: Asymmetry 2007, 18, 527-536.

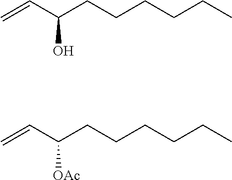

### Example 8

### (R)-tert-butyldimethyl(non-1-en-3-yloxy)silane

To a solution of (R)-oct-1-en-3-ol (0.35 g, 2.73 mmol) in CH₂Cl₂ (6.1 mL), imidazole (0.372 g, 5.46 mmol) and TBSCl (0.618 g, 4.10 mmol) were added. After 1 hour, the reaction was poured over water, extracted 3× with CH₂Cl₂ and subsequently dried over Na₂SO₄. The organic layers were then concentrated and purified via silica gel chromatography (hexanes) to yield (R)-tert-butyldimethyl(oct-1-en-3-yloxy)silane (0.630 g, 95%) as a colorless oil.

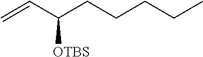 [SMILES: C=C[C@@H](CCCCC)O[Si](C)(C)C(C)(C)C]

¹H NMR (500 MHz, CDCl₃) δ=5.85-5.76 (m, 1H), 5.13 (dd, J=1.7, 17.2 Hz, 1H), 5.01 (dd, J=1.1, 10.3 Hz, 1H), 4.11-4.04 (m, 1H), 1.59-1.39 (m, 2H), 1.39-1.21 (m, 6H), 0.91-0.87 (m, 12H), 0.05 (d, J=9.7 Hz, 6H) ppm.

IR (neat) 2955, 2928, 2856, 1644, 1462, 1250, 1079, 833, 772 cm⁻¹.

### Example 9

### (S)-tert-butyl-dimethyl(oct-1-en-3-yloxy)silane

NaOH (0.169 g, 3.22 mmol) in MeOH (4 mL) was added to a solution of (S)-3-acetoxy-1-octene (0.6 g, 3.52 mmol) in MeOH (4 mL) cooled to 0° C. After 90 minutes, the reaction was poured over saturated aqueous NH₄Cl and extracted 3× with EtOAC. The combined organic layers were dried over Na₂SO₄ and concentrated to yield (S)-oct-1-en-3-ol (0.411 g, 91%) which was then used without further purification. The concentrated (S)-oct-1-en-3-ol (0.4 g, 3.12 mmol) was dissolved in CH₂Cl₂ (6.9 mL) and then imidazole (0.425 g, 6.24 mmol) and TBSCl (0.705 g, 4.68 mmol) were added. After 1 hour, the reaction was poured over water and extracted 3× with CH₂Cl₂ and subsequently dried over Na₂SO₄. The organic layers were then concentrated and purified via silica gel chromatography (hexanes) to yield (S)-tert-butyldimethyl(oct-1-en-3-yloxy)silane (0.650 g, 86%) as a colorless oil.

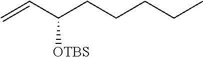 [SMILES: C=C[C@H](CCCCC)O[Si](C)(C)C(C)(C)C]

¹H NMR (500 MHz, CDCl₃) δ=5.84-5.76 (m, 1H), 5.13 (dd, J=1.7, 17.2 Hz, 1H), 5.02 (dd, J=1.7, 10.3 Hz, 1H), 4.10-4.05 (m, 1H), 1.58-1.21 (m, 8H), 0.92-0.85 (m, 12H), 0.06 (s, 3H), 0.04 (s, 3H) ppm.

IR (neat) 2955, 2928, 2856, 1644, 1462, 1250, 1079, 833, 772 cm⁻¹.

### Example 10

### (S)-TBS-Protected Product (Alkene Metathesis)

In a glove box, Hoveyda-Grubbs II catalyst (approximately 2.5 mg, 0.0039 mmol) was quickly added to a microwave vial. The vial was sealed prior to removal from the glove box. To a small vial, methyl 5-((3aS,5R,6R,6aS)-5-hydroxy-6-vinyl-1,3a,4,5,6,6a-hexahydropentalen-2-yl)pentanoate and methyl 5-((3aR,5S,6S,6aR)-5-hydroxy-6-vinyl-1,3a,4,5,6,6a-hexahydropentalen-2-yl)pentanoate (9.4 mg, 0.036 mmol) and (S)-tert-butyldimethyl(oct-1-en-3-yloxy)silane (175 mg, 0.72 mmol) were added with a small amount of CH₂Cl₂ (0.2 mL). This mixture was then added to the vial containing the catalyst and the small vial was rinsed with CH₂Cl₂ (0.1 mL). The reaction was covered with foil and left stirring at room temperature. After 3 hours, the reaction mixture was purified by silica gel chromatography (9:1, hexanes, EtOAc) which yielded methyl 5-((3 aS,5R,6R,6aS)-6-4S,E)-3-((tert-butyldimethylsilyl)oxy)oct-1-en-1-yl)-5-hydroxy-1,3a,4,5,6,6a-hexahydropentalen-2-yl)pentanoate and methyl 5-((3aR,5S,6S,6aR)-6-((S,E)-3-((tert-butyldimethylsilyl)oxy)oct-1-en-1-yl)-5-hydroxy-1,3a,4,5,6,6a-hexahydropentalen-2-yl)pentanoate (9.4 mg, 55%) as a colorless oil.

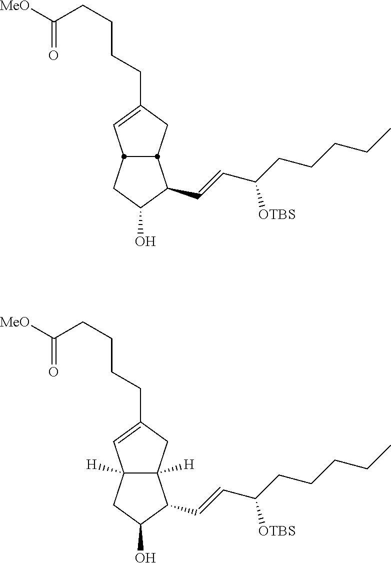 [SMILES: CCCCC[C@@H](/C=C/[C@@H]1[C@H]2CC(CCCCC(=O)OC)=C[C@H]2C[C@H]1O)O[Si](C)(C)C(C)(C)C ; CCCCC[C@@H](/C=C/[C@H]1[C@@H]2CC(CCCCC(=O)OC)=C[C@@H]2C[C@@H]1O)O[Si](C)(C)C(C)(C)C]

¹H NMR (500 MHz, CDCl₃) δ=5.59-5.51 (m, 1H), 5.49-5.40 (m, 1H), 5.32-5.27 (m, 1H), 4.11-4.04 (m, 1H), 3.81-3.71 (m, 1H), 3.68 (s, 3H), 3.06-2.97 (m, 1H), 2.46-2.38 (m, 1H), 2.33 (m, 4H), 2.08-1.96 (m, 3H), 1.96-1.88 (m, 1H), 1.67-1.22 (m, 14H), 0.93-0.89 (m, 12H), 0.06 (s, 3H), 0.04 (s, 3H) ppm.

¹³C NMR (125 MHz, CDCl₃) δ=174.3 (2C), 141.6 (2C), 136.4, 136.3, 131.0, 130.6, 128.4, 128.3, 73.7, 73.5, 58.3, 58.2, 51.6 (2C), 45.8 (2C), 44.4 (2C), 39.9, 39.8, 39.6, 39.5, 38.6, 38.5, 34.0 (2C), 31.8 (2C), 30.7 (2C), 27.3 (2C), 26.0 (8C), 25.3 (2C), 24.8 (2C), 22.7 (2C), 18.4 (2C), 14.1 (2C), −4.1 (2C), −4.6 (2C) ppm.

### Example 11

### (R)-TBS-Protected Product (Alkene Metathesis)

In a glove box, Hoveyda-Grubbs II catalyst (approximately 2.5 mg, 0.0039 mmol) was quickly added to a microwave vial. The vial was sealed prior to removal from the glove box. To a small vial, methyl 5-((3aS,5R,6R,6aS)-5-hydroxy-6-vinyl-1,3a,4,5,6,6a-hexahydropentalen-2-yl)pentanoate and methyl 5-((3aR,5S,6S,6aR)-5-hydroxy-6-vinyl-1,3a,4,5,6,6a-hexahydropentalen-2-yl)pentanoate (10 mg, 0.038 mmol) and (R)-tert-butyldimethyl(oct-1-en-3-yloxy)silane (184 mg, 0.76 mmol) were added with a small amount of CH₂Cl₂ (0.2 mL). This mixture was then added to the vial containing the catalyst and the small vial was rinsed with CH₂Cl₂ (0.1 mL). The reaction was covered with foil and left stirring at room temperature. After 2 hours, the reaction mixture was purified by silica gel chromatography (9:1, hexanes, EtOAc) which yielded methyl 5-((3 aS,5R,6R,6aS)-6-((R,E)-3-((tert-butyldimethylsilyl)oxy)oct-1-en-1-yl)-5-hydroxy-1,3a,4,5,6,6a-hexahydropentalen-2-yl)pentanoate and methyl 5-((3aR,5S,6S,6aR)-6-((R,E)-3-((tert-butyldimethylsilyl)oxy)oct-1-en-1-yl)-5-hydroxy-1,3a,4,5,6,6a-hexahydropentalen-2-yl)pentanoate (8.7 mg, 48%) as a colorless oil.

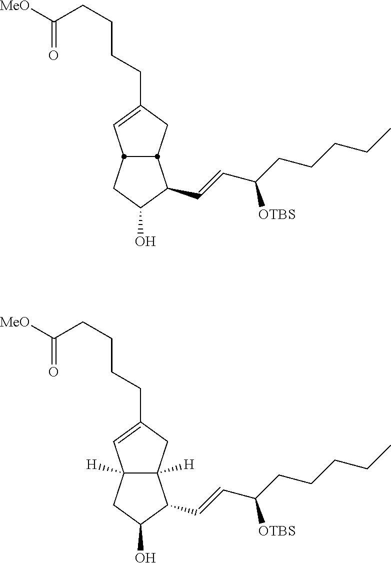 [SMILES: CCCCC[C@H](/C=C/[C@@H]1[C@H]2CC(CCCCC(=O)OC)=C[C@H]2C[C@H]1O)O[Si](C)(C)C(C)(C)C ; CCCCC[C@H](/C=C/[C@H]1[C@@H]2CC(CCCCC(=O)OC)=C[C@@H]2C[C@@H]1O)O[Si](C)(C)C(C)(C)C]

¹H NMR (500 MHz, CDCl₃) δ=5.58-5.51 (m, 1H), 5.48-5.40 (m, 1H), 5.31-5.28 (m, 1H), 4.11-4.04 (m, 1H), 3.83-3.71 (m, 1H), 3.68 (s, 3H), 3.06-2.96 (m, 1H), 2.47-2.38 (m, 1H), 2.37-2.26 (m, 4H), 2.07-1.96 (m, 3H), 1.96-1.88 (m, 1H), 1.67-1.22 (m, 14H), 0.92-0.86 (m, 12H), 0.06 (s, 3H), 0.04 (s, 3H) ppm.

¹³C NMR (125 MHz, CDCl₃) δ=174.3 (2C), 141.6 (2C), 136.4, 136.3, 131.0, 130.6, 128.4, 128.3, 73.7, 73.5, 58.3, 58.2, 51.6 (2C), 45.8 (2C), 44.4 (2C), 39.9, 39.8, 39.6, 39.5, 38.6, 38.5, 34.0 (2C), 31.8 (2C), 30.7 (2C), 27.3 (2C), 26.0 (8C), 25.3 (2C), 24.8 (2C), 22.7 (2C), 18.4 (2C), 14.1 (2C), −4.1 (2C), −4.6 (2C) ppm.

### Example 12

### (S)-Final Product

To a solution of methyl 5-((3aS,5R,6R,6aS)-6-((S,E)-3-((tert-butyldimethylsilyl)oxy)oct-1-en-1-yl)-5-hydroxy-1,3a,4,5,6,6a-hexahydropentalen-2-yl)pentanoate and methyl 5-((3 aR,5S,6S,6aR)-6-4S,E)-3-((tert-butyldimethylsilyl)oxy)oct-1-en-1-yl)-5-hydroxy-1,3a,4,5,6,6a-hexahydropentalen-2-yl)pentanoate (16.9 mg, 0.035 mmol) in THF (0.35 mL), TBAF (70 μL, 0.07 mmol) was added slowly. After 42 hours, more TBAF (35 μL, 0.035 mmol) was added; however no change was observed via TLC so at 48 hours, the reaction was poured over water and extracted 3× with EtOAc. The combined organic layers were then washed with brine, extracted 3× with EtOAc and dried using Na₂SO₄. The organic layers were then concentrated and purified using silica gel chromatography (2:8, hexanes:EtOAc) which yielded methyl 5-((3aS,5R,6R,6aS)-5-hydroxy-6-((S,E)-3-hydroxyoct-1-en-1-yl)-1,3a,4,5,6,6a-hexahydropentalen-2-yl)pentanoate and methyl 5-((3 aR,5S,6S,6aR)-5-hydroxy-6-((S,E)-3-hydroxyoct-1-en-1-yl)-1,3a,4,5,6,6a-hexahydropentalen-2-yl)pentanoate (1.5 mg, 16%) as a colorless oil.

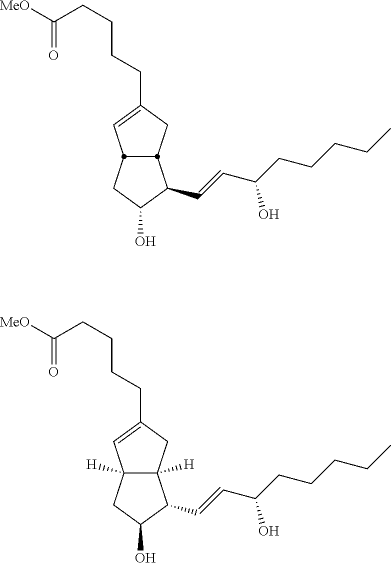 [SMILES: CCCCC[C@H](O)/C=C/[C@@H]1[C@H]2CC(CCCCC(=O)OC)=C[C@H]2C[C@H]1O ; CCCCC[C@H](O)/C=C/[C@H]1[C@@H]2CC(CCCCC(=O)OC)=C[C@@H]2C[C@@H]1O]

¹H NMR (500 MHz, CDCl₃) δ=5.60 (dd, J=6.1, 15.5 Hz, 2H), 5.30 (s, 1H), 4.12 (q, J=7.3 Hz, 1H), 3.78 (q, J=9.2 Hz, 1H), 3.68 (s, 3H), 3.05-2.99 (m, 1H), 2.44 (d, J=8.7 Hz, 1H), 2.37-2.28 (m, 4H), 2.08-2.00 (m, 3H), 1.96 (q, J=9.4 Hz, 1H), 1.66-1.25 (m, 15H), 0.91-0.88 (m, 3H) ppm.

¹³C NMR (125 MHz, CDCl₃) δ=174.1, 141.4, 135.4, 132.4, 128.3, 73.0, 58.1, 51.5, 45.6, 44.4, 39.7 (2C), 37.4, 33.9, 31.7, 30.9, 30.6, 27.2, 25.2, 24.7, 22.6, 14.0 ppm.

### Example 13

### (R)-Final Product

To a solution of methyl 5-((3aS,5R,6R,6aS)-6-((R,E)-3-((tert-butyldimethylsilyl)oxy)oct-1-en-1-yl)-5-hydroxy-1,3a,4,5,6,6a-hexahydropentalen-2-yl)pentanoate and methyl 5-((3 aR,5S,6S,6aR)-6-4R,E)-3-((tert-butyldimethylsilyl)oxy)oct-1-en-1-yl)-5-hydroxy-1,3a,4,5,6,6a-hexahydropentalen-2-yl)pentanoate (13 mg, 0.027 mmol) in THF (0.27 mL), TBAF (54 μL, 0.054 mmol)) was added slowly. After 24 hours, additional TBAF (27 μL, 0.027 mmol) was added. At 48 hours, ethoxytrimethylsilane (84 μL, 0.54 mmol) was added to quench the excess TBAF. After 45 minutes of additional stirring, the reaction was concentrated and directly purified using silica gel chromatography (6:4, hexanes:EtOAc) to yield methyl 5-((3aS,5R,6R,6aS)-5-hydroxy-6-((R,E)-3-hydroxyoct-1-en-1-yl)-1,3a,4,5,6,6a-hexahydropentalen-2-yl)pentanoate and methyl 5-((3aR,5S,6S,6aR)-5-hydroxy-6-((R,E)-3-hydroxyoct-1-en-1-yl)-1,3a,4,5,6,6a-hexahydropentalen-2-yl)pentanoate (1.4 mg, 14%) as a colorless oil.

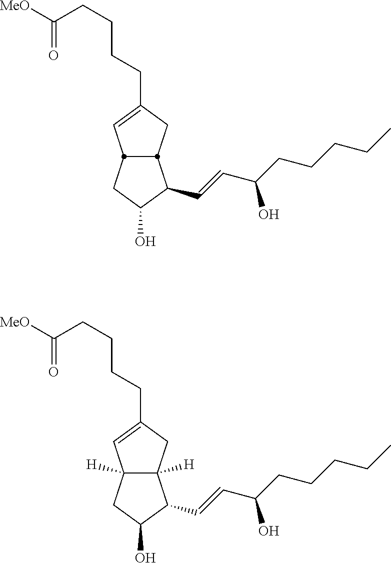 [SMILES: CCCCC[C@@H](O)/C=C/[C@@H]1[C@H]2CC(CCCCC(=O)OC)=C[C@H]2C[C@H]1O ; CCCCC[C@@H](O)/C=C/[C@H]1[C@@H]2CC(CCCCC(=O)OC)=C[C@@H]2C[C@@H]1O]

¹H NMR (500 MHz, CDCl₃) δ=5.60 (dd, J=6.1, 15.5 Hz, 2H), 5.30 (s, 1H), 4.12 (q, J=7.3 Hz, 1H), 3.78 (q, J=9.2 Hz, 1H), 3.68 (s, 3H), 3.05-2.99 (m, 1H), 2.44 (d, J=8.7 Hz, 1H), 2.37-2.28 (m, 4H), 2.08-2.00 (m, 3H), 1.96 (q, J=9.4 Hz, 1H), 1.66-1.25 (m, 15H), 0.91-0.88 (m, 3H) ppm.

¹³C NMR (125 MHz, CDCl₃) δ=174.1, 141.4, 135.4, 132.4, 128.3, 73.0, 58.1, 51.5, 45.6, 44.4, 39.7 (2C), 37.4, 33.9, 31.7, 30.9, 30.6, 27.2, 25.2, 24.7, 22.6, 14.0 ppm.

### Example 14

### Octene Product (Alkene Metathesis)

In a glove box, Hoveyda-Grubbs II catalyst (approximately 1 mg, 0.00159 mmol) was quickly added to a microwave vial. The vial was sealed prior to removal from the glove box. To a small vial, methyl 5-((3aS,5R,6R,6aS)-5-hydroxy-6-vinyl-1,3a,4,5,6,6a-hexahydropentalen-2-yl)pentanoate and methyl 5-((3aR,5S,6S,6aR)-5-hydroxy-6-vinyl-1,3a,4,5,6,6a-hexahydropentalen-2-yl)pentanoate of Example 6 (4.2 mg, 0.0159 mmol) and 1-octene (50 μL, 0.318 mmol) were added with a small amount of CH₂Cl₂ (<0.1 mL). This mixture was then added to the vial containing the catalyst and the small vial was rinsed with CH₂Cl₂ (0.1 mL). The reaction was covered with foil and left stirring at room temperature. After 3 hours, the reaction mixture was purified by silica gel chromatography (9:1, hexanes:EtOAc) which yielded methyl 5-((3aS,5R,6R,6aS)-5-hydroxy-6-((E)-oct-1-en-1-yl)-1,3a,4,5,6,6a-hexahydropentalen-2-yl)pentanoate and methyl 5-((3 aR,5S,6S,6aR)-5-hydroxy-6-((E)-oct-1-en-1-yl)-1,3a,4,5,6,6a-hexahydropentalen-2-yl)pentanoate (2.6 mg, 44%).

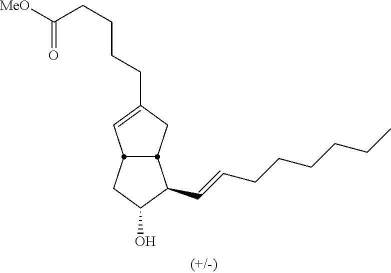 [SMILES: CCCCCC/C=C/[C@@H]1[C@H]2CC(CCCCC(=O)OC)=C[C@H]2C[C@H]1O]

¹H NMR (500 MHz, CDCl₃) δ=5.54 (dt, J=6.7, 15.1 Hz, 1H), 5.33-5.27 (m, 2H), 3.76-3.70 (m, 1H), 3.68 (s, 3H), 3.03-2.96 (m, 1H), 2.45-2.39 (m, 1H), 2.33 (t, J=8.0 Hz, 3H), 2.31-2.27 (m, 2H), 2.09-1.98 (m, 5H), 1.87 (q, J=9.3 Hz, 1H), 1.67-1.60 (m, 3H), 1.47 (quin, J=8.0 Hz, 2H), 1.41-1.24 (m, 10H), 0.89 (t, J=6.9 Hz, 3H) ppm.

¹³C NMR (125 MHz, CDCl₃) δ=174.2, 141.4, 133.3, 131.0, 128.3, 58.7, 51.5, 45.6, 44.4, 39.7, 39.4, 33.9, 32.7, 31.7, 30.6, 29.5, 28.8, 27.2, 24.7, 22.6, 14.1 ppm.

Various embodiments of the invention have been described in fulfillment of the various objects of the invention. It should be recognized that these embodiments are merely illustrative of the principles of the present invention. Numerous modifications and adaptations will be readily apparent to those skilled in the art without departing from the spirit and scope of the invention.

That which is claimed is:

1. A method of providing clinprost, isocarbacylcin or an analog thereof comprising:
providing an ester of formula (1): 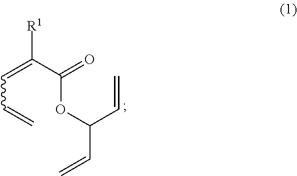
performing decarboxylation with concomitant allylic transposition of the ester of formula (1) to provide a reaction product mixture comprising a tetraene of formula (2): 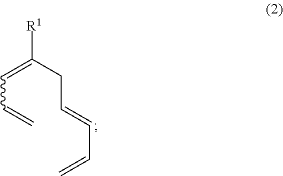
performing cyclocarbonylation on the tetraene of formula (2) and reducing a resulting ketone reaction product to provide a compound of formula (3): 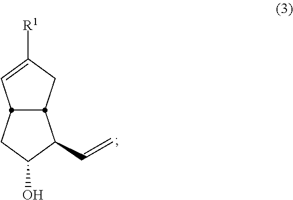
and
performing cross-metathesis with the compound of formula (3) and an alkene to provide a compound of formula (4): 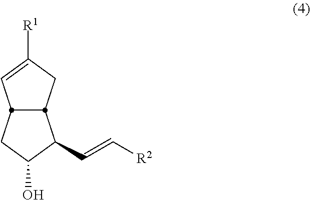
wherein R¹ is selected from the group consisting of -alkyl-C(O)OR³ and -alkyl-OR³,
wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.

2. The method of claim 1, wherein the decarboxylative allylation is performed with a palladium catalyst.

3. The method of claim 1, wherein the cyclocarbonylation is performed with a rhodium catalyst.

4. The method of claim 1, wherein the cross-metathesis is performed with a ruthenium catalyst.

5. The method of claim 1, wherein the alkene is selected from the group consisting

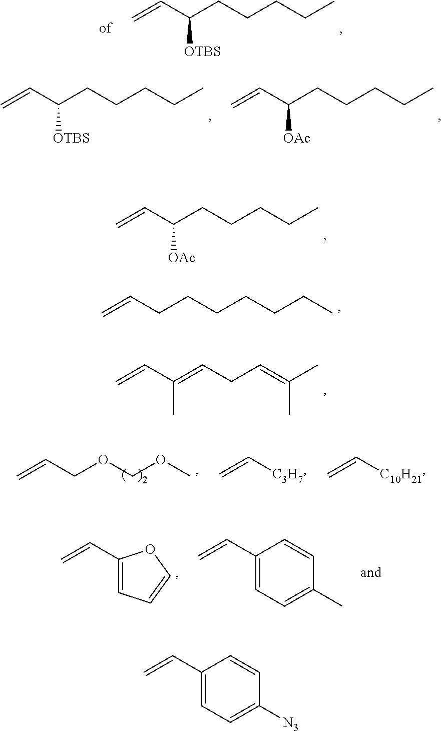

6. A compound of formula (4): 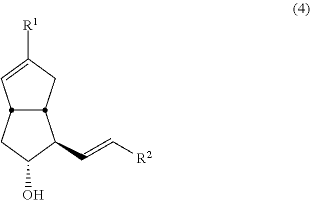
wherein R¹ is selected from the group consisting of —C(O)OR³, -alkyl-C(O)OR³ and -alkyl-OR³, wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.

7. A method of providing clinprost or isocarbacylcin analogs comprising:
providing an ester of formula (6): 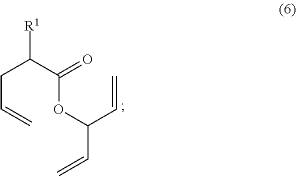
performing decarboxylation with concomitant allylic transposition of the ester of formula (6) to provide a reaction product mixture comprising a tetraene of formula (7): 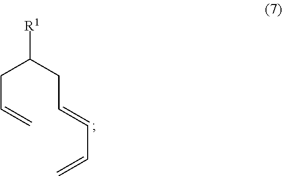
performing cyclocarbonylation on the tetraene of formula (7) and reducing a resulting ketone reaction product to provide a compound of formula (8): 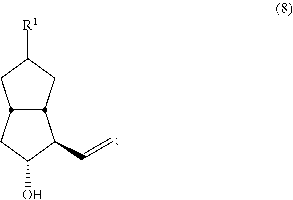
and
performing cross-metathesis with the compound of formula (8) and an alkene to provide a compound of formula (9): 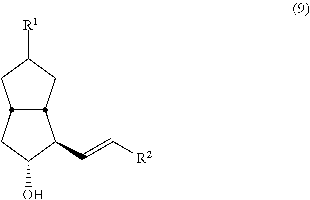
wherein R¹ is selected from the group consisting of —C(O)OR³, -alkyl-C(O)OR³ and -alkyl-OR³, wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.

8. The method of claim 7, wherein the alkene is selected from the group consisting

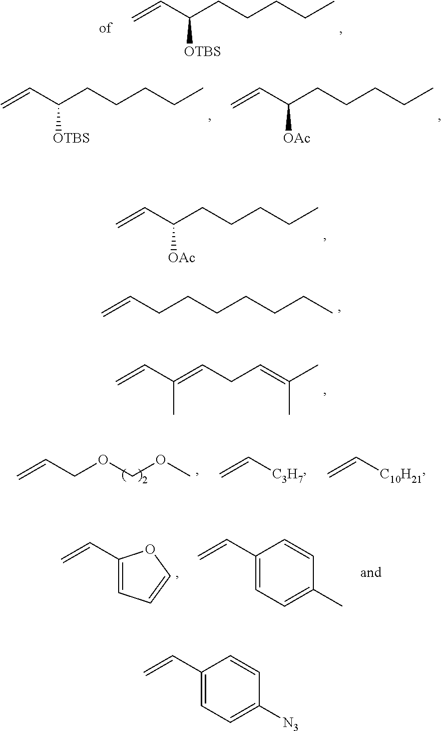

9. A compound of formula (9): 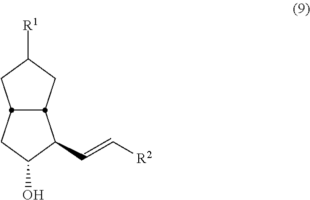
wherein R¹ is selected from the group consisting of —C(O)OR³, -alkyl-C(O)OR³ and -alkyl-OR³, wherein R³ is hydrogen or alkyl and wherein R² is selected from the group consisting of -alkyl, -alkenyl, -alkynyl, -cycloalkyl, -aryl, -heteroaryl, -heterocyclyl, -alkoxy, -alkyl-aryl, -alkyl-heteroaryl and —R⁴—O—R⁵—O—R⁶ wherein R⁴, R⁵ and R⁶ are independently selected from the group consisting of alkyl and alkenyl and wherein the alkyl, alkenyl, alkynyl, cycloalkyl, aryl, heteroaryl and heterocyclyl of R² are optionally substituted with -hydroxy, -alkyl, -alkoxy or —N₃.
